SEQUENCE LISTING (3 sequences)

SEQ ID NO: 1 - protein, 13 aa, Feline immunodeficiency virus
  MISC_FEATURE: An env polypeptide containing a variant of amino acids 696-707 of native FIV env sequence with a non-native N-terminal cysteine residue.
CELGCNQNQFFCK

SEQ ID NO: 2 - protein, 13 aa, Feline immunodeficiency virus
  MISC_FEATURE: An env polypeptide containing a variant of amino acids 696-707 of native FIV env sequence with a non-native N-terminal cysteine residue.
CELGSNQNQFFSK

SEQ ID NO: 3 - protein, 45 aa, Feline immunodeficiency virus
  MISC_FEATURE: An env polypeptide containing a variant of amino acids 696-707 of native FIV env sequence.
ELGSNQNQFFSKVPPELWKRYNKSKSKSKSKNRWEWRPDFESEKC

(19) United States
(12) Patent Application Publication
(10) Pub. No.: US 2006/0003445 A1
(43) Pub. Date: Jan. 5, 2006

(54) Method and device for detecting feline immunodeficiency virus

(75) Inventors: Randall G. Groat, Freeport, ME (US); Quentin J. Tonelli, Portland, ME (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: IDEXX Laboratories, Inc., Westbrook, ME (US)

(21) Appl. No.: 11/075,958

(22) Filed: Mar. 9, 2005

Related U.S. Application Data
(60) Provisional application No. 60/584,573, filed on Jun. 30, 2004.

(51) Int. Cl. C12Q 1/68; G01N 33/53; A61K 39/12; C12N 5/06; C12N 5/16

(52) U.S. Cl. 435/345; 435/7.1; 435/6

(57) ABSTRACT

A method and device for determining a feline immunodeficiency virus infection or vaccination in an animal. The method is an assay involving contacting a biological sample from a felid with an env FIV polypeptide and determining the binding of antibodies in the sample to the polypeptide. The assay is optimized by adjusting the concentration of the sample and reagents, and the time and temperature of the incubation, so that antibodies in samples from vaccinated animals do not substantially bind to the polypeptide.

### CROSS-REFERENCE TO RELATED APPLICATION

This application claims the benefit of U.S. Provisional Patent Application Ser. No. 60/584,573 filed Jun. 30, 2004.

### FIELD OF THE INVENTION

The invention is related to the detection of antibodies directed to Feline Immunodeficiency Virus.

### BACKGROUND OF THE INVENTION

Feline immunodeficiency virus (FIV), formerly called feline T-lymphotrophic lentivirus, was first isolated in 1986 from a large multiple cat household in Petaluma, Calif. (Pederson et al., Science (1987) 235:790). FIV infects cats to produce an AIDS-like syndrome. Although FIV is morphologically and pathologically similar to the human immunodeficiency virus (HIV), it has been shown to be antigenically distinct from HIV. Like HIV, once a cat becomes infected with FIV, the disease progresses from a primary infection (viraemia, fever, general lymphadenitis) to a lengthy asymptomatic phase, followed by severe impairment in immune function caused by a reduction in CD4 lymphocytes, and resulting in heightened susceptibility to secondary infections and ultimately death.

FIV has been classified as a member of the subfamily Lentiviridae in the family Retroviridae, the family that includes human and simian immunodeficiency viruses, equine infectious anaemia, maedi visna of sheep and caprinearthritis encephalitis viruses (CAEV). The genome of FIV is organized like other lentiviruses with three long open reading frames corresponding to gag, pol and env (Talbott et al., Proc. Natl. Acad. Sci. (1989) 86:5743; Olmsted et al., Proc. Natl. Acad. Sci. (1989) 86:2448). The gag gene codes for the major structural components of the virus, the env gene codes for the envelope glycoprotein, and the pol gene codes for the polymerase protein.

The gag gene is expressed as a 55 kD polyprotein that is processed into three subunits: a p15 matrix protein, a p24 capsid protein, and a p10 nucleocapsid protein. The pol gene encodes three proteins: the protease, reverse transcriptase and a p14.6 protein of unknown function. Autoprocessing by the protease portion of the gene gives rise to all three proteins of the pol region. Additionally, the protease is responsible for the processing of the gag precursor. The pol gene is expressed as a gag-pol fusion protein. The envelope gene is expressed as a 160 kD glycoprotein, gp160. The antigenicity of the FIV core proteins is similar to other lentiviruses.

Several independent viral isolates have been prepared across the world, and a certain number of studies have been carried out in order to demonstrate the structure of the isolated strains: the American strain Petaluma, Talbott et al. Natl. Acad. Sci. USA, 1989, 86, 5743-5747; Philipps et al., J. Virol., 1990, 64, 10, 4605-4613), the Japanese strains (the TM1 and TM2 strains), Miyazawa et al., Arch. Virol., 1989, 108, 59-68, and the Swiss isolates (FIVZ1 and FIVZ2), Morikawa et al., Virus Research, 1991, 21, 53-63.

The nucleotide sequences of three proviral clones derived from American FIV isolates (Petaluma strain) have been described (clones FIV34TF10, FIV14 and isolate PPR) (Olmsted, et al. 1989; Philipps et al., 1990; Talbott et al., 1989) and compared with two Swiss isolates (Morikawa et al. 1991). This comparison led Morikawa et al. to specify the presence of certain conserved regions and certain variable regions within the env gene of FIV. French strains have also been isolated (strains Wo and Me) (Moraillon et al., 1992, Vet. Mic., 31, 41-45).

The virus replicates optimally in blood mononuclear cells and has a tropism for T-lymphocytes, peritoneal macrophage, brain macrophage and astrocytes. In common with other retroviruses, the genetic material of FIV is composed of RNA and the production of a DNA copy of the viral RNA is an essential step in the replication of FIV in the host. This step requires the enzyme reverse transcriptase that is carried into the host by the invading virus. The DNA version of the viral genome is inserted into the genetic material of infected host cells in which it continues to reside as a provirus. This provirus is replicated every time the cell divides and can code for the production of new virus particles. Cells infected with FIV remain infected for the duration of their lifespan.

The virus appears to be spread naturally by horizontal transmission, predominantly by bite wounds from an infected cat as these animals shed appreciable amounts of virus in saliva (Yamamoto et al., Am. J. Vet. Res. 1988, 8:1246). Vertical transmission has been reported, but is rare.

Current diagnostic screening tests for FIV infection detect serum antibody (Ab) to FIV. Virus detection kits are also available but not as prevalent. A number of diagnostic tests are available to determine the presence of FIV antibody in infected animals. For example, PetChek® FIV Ab test kit and the SNAP® Combo FeLV Ag/FIV Ab test kit (IDEXX Laboratories, Westbrook, Me.) are immunoassay based diagnostic tests for FIV infection.

Detecting FIV infection is becoming increasingly important as studies reveal FIV infection is widespread worldwide. As vaccines have been developed in attempt to combat the disease, it is even more important to be able to detect the effectiveness of a vaccine and to discriminate between vaccinated cats versus naturally infected cats.

### SUMMARY OF THE INVENTION

A method for detecting antibodies to Feline Immunodeficiency Virus (FIV) in a biological sample, the method comprising contacting the biological sample with an FIV env polypeptide and detecting whether the polypeptide substantially binds to an antibody in the sample, wherein the reaction conditions are optimized so that the method will detect FIV antibodies in a sample from animals that have been naturally infected but the method will not detect antibodies in a sample from animals that have been vaccinated.

In various aspects of the invention, the method is optimized by diluting the sample, by adjusting the concentration of the polypeptide, by adjusting the temperature of the reaction, and/or by adjusting the time of the reaction.

### BRIEF DESCRIPTION OF THE FIGURES

FIGS. 1A-1C and 2A-2C show the results of immunoassays on serial dilutions of serum samples. FIV antibodies that substantially bound to FIV env polypeptide were detected.

### DETAILED DESCRIPTION

Before describing the present invention in detail, a number of terms will be defined. As used herein, the singular forms “a,” “an”, and “the” include plural referents unless the context clearly dictates otherwise.

As used herein, the term “polypeptide” refers to a compound of a single chain or a complex of two or more chains of amino acid residues linked by peptide bonds. The chain(s) may be of any length. A protein is a polypeptide and the terms are used synonymously. Also included within the scope of the invention are functionally equivalent variants and fragments of FIV polypeptides. The polypeptide is capable of binding one or more antibodies specific for the polypeptide.

Polypeptides derived from FIV include any region of the of the FIV proteome including for example, portions of the gag and env regions and mimitopes thereof. U.S. Pat. Nos. 5,648,209, 5,591,572, and 6,458,528, which are incorporated by reference herein in their entirety, describe FIV polypeptides derived from the FIV env and gag proteins. These peptides, and others like them, from the env and gag proteins, are suitable for use in the methods of the present invention. An example of a suitable env polypeptide is amino acids 696-707 of native FIV env sequence, shown here with a non-native native N-terminal cysteine residue:

[SEQ ID NO:1]

CELGCNQNQFFCK

Other useful polypeptides include variants of SEQ ID NO:1 including the following:

[SEQ ID NO:2]

CELGSNQNQFFSK

[SEQ ID NO:3]

ELGSNQNQFFSKVPPELWKRYNKSKSKSKSKNRWEWRPDFESEKC

“Binding specificity” or “specific binding” refers to the substantial recognition of a first molecule for a second molecule, for example a polypeptide and a polyclonal or monoclonal antibody, or an antibody fragment (e.g. a Fv, single chain Fv, Fab′, or F(ab′)2 fragment) specific for the polypeptide. “Substantial binding” or “substantially bind” refer to an amount of specific binding or recognizing between molecules in an assay mixture under particular assay conditions. In its broadest aspect, substantial binding relates to the difference between a first molecule's incapability of binding or recognizing a second molecule, and the first molecules capability of binding or recognizing a third molecule, such that the difference is sufficient to allow a meaningful assay to be conducted distinguishing substantial binding under a particular set of assay conditions, which includes the relative concentrations of the molecules. In another aspect, one molecule is substantially incapable of binding or recognizing another molecule in a cross-reactivity sense where the first molecule exhibits a reactivity for a second molecule that is less than 25%, preferably less than 10%, more preferably less than 5% of the reactivity exhibited toward a third molecule under a particular set of assay conditions, which includes the relative concentration and incubation of the molecules. Specific binding can be tested using a number of widely known methods, e.g, an immunohistochemical assay, an enzyme-linked immunosorbent assay (ELISA), a radioimmunoassay (RIA), or a western blot assay.

Animals infected with FIV are felids, which is to be understood to include all members of the order Felidae, including domestic cats, lions, tigers, jaguars, leopards, puma, ocelots, etc. As used herein, the terms “cat” or “animal” is a reference to all felids.

A “biological sample” refers to a sample from an animal subject including saliva, whole blood, serum, plasma or other sample known to contain FIV antibodies

Vaccines for FIV are described, for example, in U.S. Pat. Nos. 6,667,295, 5,833,993, 6,447,993, 6,254,872 and 6,544,528, and published U.S. patent application 20040096460, each of which is incorporated herein by reference in their entirety. U.S. Pat. Nos. 6,447,993 and 6,254,872 describe vaccines that are prepared from cell free-viral isolates of different FIV subtypes or a combination of cell lines each infected with different prototype FIV virus from a different subtype. U.S. Pat. No. 5,833,933 describes vaccines containing DNA sequences encoding FIV gag protein and FIV env protein. These vaccines include an expression system for expressing the sequences. One available vaccine is FEL-O-VAX® FIV (Fort Dodge Animal Health, Overland Park, Kans.).

Biological samples from animals that have been vaccinated against an FIV infection have the potential for producing a positive result in a test for an FIV infection due to the presence of antibodies produced in response to the vaccine. In one aspect, the invention provides for a method of distinguishing animals that have been naturally infected with FIV from animals that have not been infected or have been vaccinated against an FIV infection.

The invention exploits differences in affinity and/or binding kinetics of anti-(FIV env) antibodies from naturally infected animals compared to those antibodies from vaccinated animals. Generally, the method includes contacting a biological sample from an animal with a solid phase having immobilized thereon an FIV env polypeptide After washing the solid phase, a labeled anti-(feline IgG) second antibody can be used to detect anti-(FIV env) antibody that binds to the FIV env polypeptide on the solid phase by procedures well known in the art of immunoassays. The method can be optimized so that the assay will detect antibodies that are an animal's immune response to a natural infection but will not detect antibodies that are the animal's immune response to a vaccination.

In one aspect, the invention provides for a method for detecting sample antibody that is a component of an animal's immune response to a FIV infection, but not to vaccination. The method includes obtaining a biological sample from an animal and contacting the sample with a solid phase having immobilized thereon an FIV env polypeptide. The solid phase is commonly a microtiter plate or a solid phase matrix of a lateral flow device, but the invention is capable of being practiced in all of formats generally known in the immunoassay arts. Attachment of the FIV env polypeptide to the solid phase can be accomplished by procedures well known to those of skill in the art of immobilization of polypeptides.

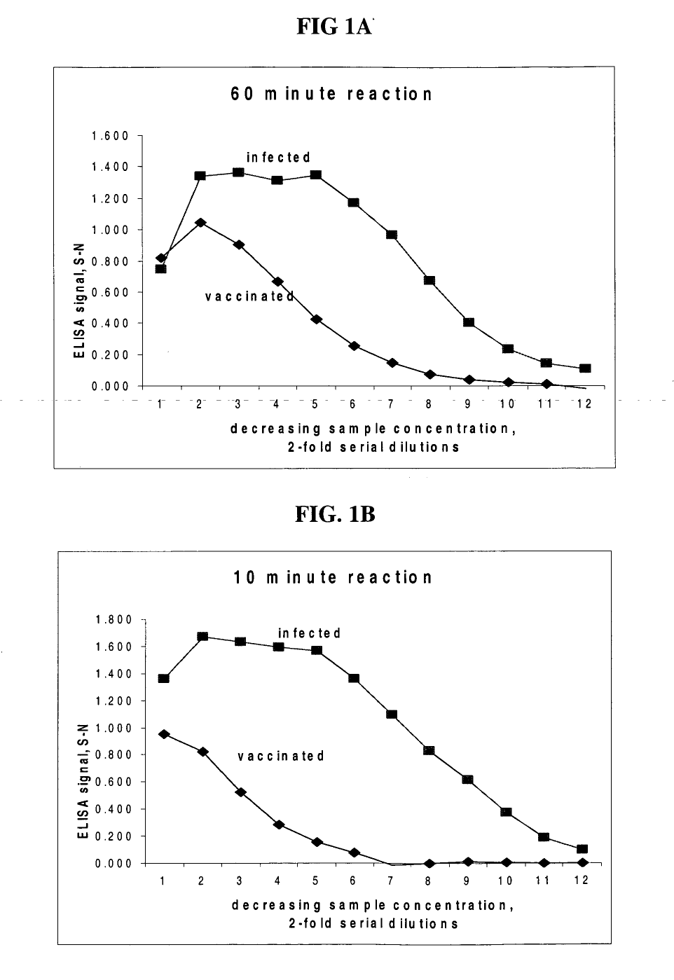

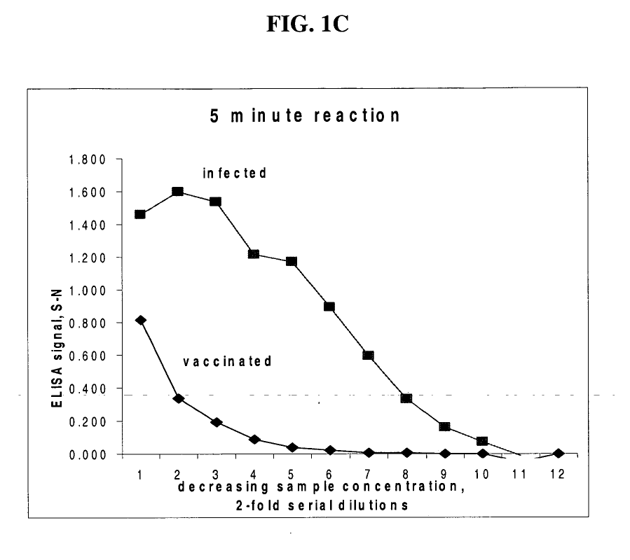

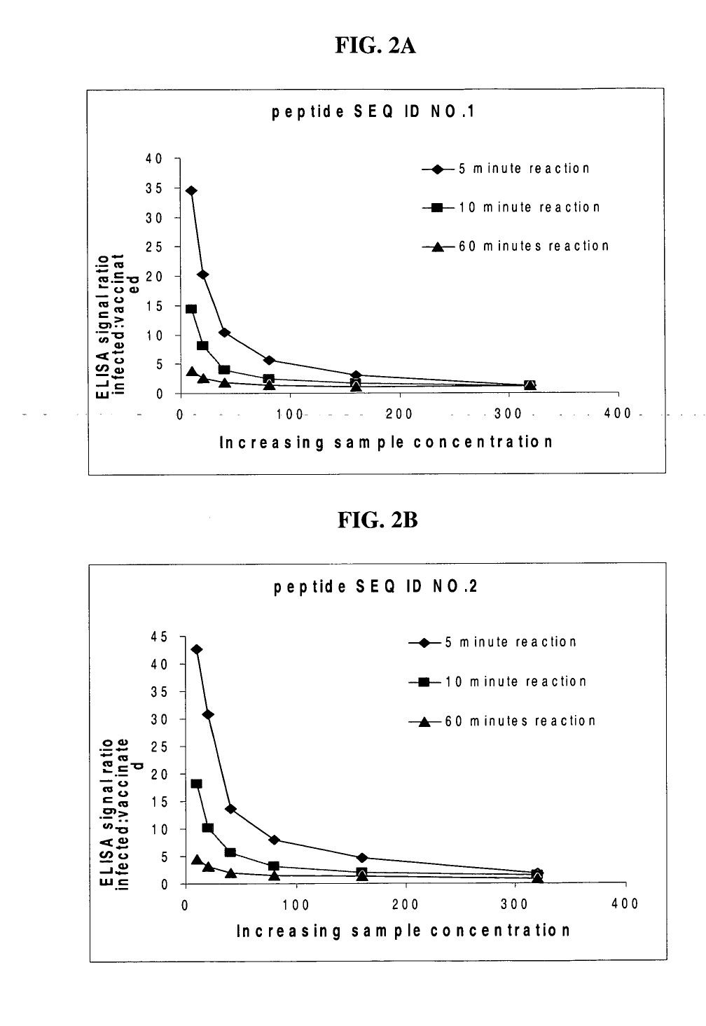

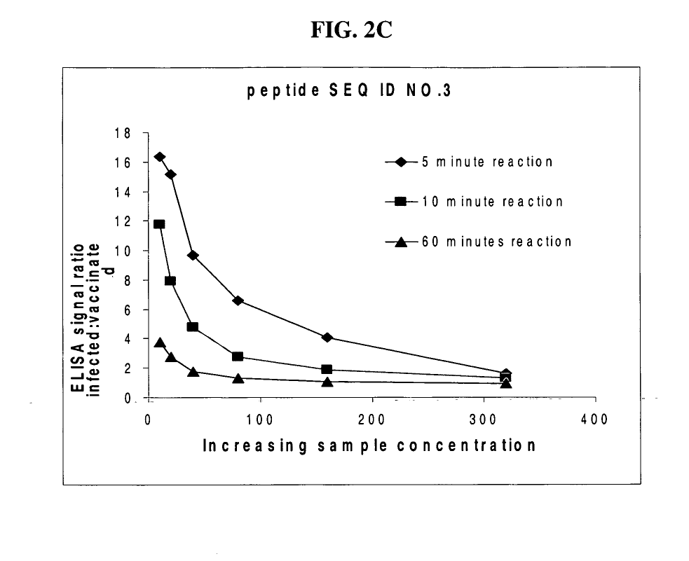

In one aspect, the method is optimized by diluting the sample. FIGS. 1 and 2 show the results of assays performed at various sample dilutions and reaction times. These FIGs show the relationship between sample dilution and assay signal (A650 nm) for reaction times of 5, 10 and 60 minutes. In FIG. 1, relative sample antibody concentration is shown in two-fold sample dilutions. In FIG. 2, sample antibody concentration (dilution) is shown relative to a 320-fold dilution of serum sample which has been assigned an arbitrary concentration of 10. As shown in FIGS. 1 and 2, when the appropriate sample dilution and reaction times are used, animals that are naturally infected with FIV can be distinguished from animals that have been vaccinated.

Because antibody detection by the method is in part determined by the concentration of the labeled anti-(feline IgG), the exact dilution at which a sample from a vaccinated animal will no longer provide a positive result depends in part on the working concentration of the conjugate. The appropriate working concentration of the conjugate can be determined based on a maximum signal for a positive control at a specific dilution and a minimal signal for a negative control at that dilution.

Once a working concentration of conjugate has been determined, the dilution at which a vaccinated sample will not provide a positive result can be determined by titrating control sera from vaccinated animals in the method of the assay.

Similar to diluting the sample, the method of the invention can be optimized by adjusting the concentration of the working conjugate. For example, a lower sample dilution should not provide a positive result for vaccinated animals when the conjugate concentration is relatively low. Likewise, a higher sample dilution should still provide a positive result for vaccinated animals when the conjugate concentration is relatively high. One of skill in the art could readily adjust the sample dilution and/or the concentration of the conjugate to optimize the method so that the assay will detect the animal's immune response to a natural infection but will not detect an animal's immune response to a vaccination.

Additional ways of optimizing the method of the invention include adjusting time and temperature of the incubation steps. In various aspects of the method of the invention, the incubation is at room temperature (approximately 20 degrees C.) and the time of the incubation is kept to the shortest period possible. As discussed herein, the method of the invention can be optimized in many ways and one of skill in the art could simultaneously adjust the dilutions, concentrations, temperatures and times used in the method to accomplish a differential detection of serum having antibodies to a FIV infection or vaccination.

The development of FIV antibodies in an animal against a vaccine is dependent upon the vaccine. For example, it has been found that animals test seropositive for antibodies against FIV p24 (gag) protein about two to four weeks after vaccination with the FEL-O-VAX® vaccine. However, animals so vaccinated may not generate persistent antibodies against one or more regions of the env protein. In contrast, naturally infected animals typically generate antibodies to both FIV gag and env proteins.

In some instances, during an initial phase following a vaccination, an animal may temporarily (transiently) produce lower levels of certain antibodies to specific FIV polypeptides as compared to those produced in response to a natural infection. These antibody levels taper off after a period of time to the point that antibody to these polypeptides is not detected after the initial phase. Generally, this amount of time is about ten to twelve weeks, but will vary between species and individual subject animals. These antibodies are not detected as a significant component of the animal's immune response to the vaccine after the initial phase.

For example, FIV gag proteins p15 and p24 may be immunogenic components of a killed whole virus FIV vaccine. It is expected that these components elicit a relatively persistent antibody response when administered to an animal. On the other hand, some vaccines may not include immunologically significant quantities of certain FIV env polypeptides or, this polypeptide has been altered in the process of virus inactivation, or presentation of this protein by vaccination differs from that for natural infection to a point where antibodies produced thereto, if any, are detected for a period of time less than antibodies to p15 and p24. Thus, while during the initial phase following vaccination, an animal may transiently produce low levels of such antibodies that bind to certain FIV env polypeptides, any such antibody production declines over a period of time and is not detected after about 12 weeks. In this example, the transiently produced antibodies are not detected as a significant component of the animal's immune response to the vaccine after a period of time.

Given that the production of detectable antibodies that are directed toward certain FIV env polypeptides usually drops off after about 12 weeks from completion of vaccination, in one aspect of the invention, the biological sample is preferably obtained from the animal that has not received an FIV vaccine within about the prior 12 weeks. If the vaccination status is unknown and the test results is positive, a retest after an additional 12 weeks can be recommended.

In one aspect of the invention, the polypeptides are immobilized on a suitable solid support. The biological sample is brought into contact with the polypeptide, to which the anti-FIV antibodies bind, if such antibodies are present in the sample. The binding may be detected by any suitable means, e.g., enzymes, radionuclides, particulates or fluorescent labels. In a suitable embodiment, the detection reagent can be associated with a polypeptide that is the same or similar to that which is used to capture anti-FIV antibodies (if present).

The polypeptides used in the invention contain at least six amino acids, usually at least nine amino acids, and more usually twelve or more amino acids found within one of the natural FIV proteins and mimitopes and functionally equivalent variants thereof.

“Functional equivalent” or “Functionally equivalent” refers to polypeptides related to or derived from the native FIV envelope (env) and viral core (gag) polypeptide sequences where the amino acid sequence has been modified by a single or multiple amino acid substitution, insertion, deletion, and also sequences where the amino acids have been chemically modified, such as amino acid analogs, but which nonetheless retain substantially equivalent function. Functionally-equivalent variants may occur as natural biological variations or may be prepared using known techniques such as chemical synthesis, site-directed mutagenesis, random mutagenesis, or enzymatic cleavage and/or ligation of amino acids. Thus, modification of the amino-acid sequence to obtain variant sequences may occur so long as the function of the polypeptide is not affected.

FIV functionally-equivalent variants within the scope of the invention may comprise conservatively substituted sequences, meaning that one or more amino acid residues of the FIV polypeptide are replaced by different residues that do not alter the secondary and/or tertiary structure of the FIV polypeptide. Such substitutions may include the replacement of an amino acid by a residue having similar physicochemical properties, such as charge density, size, configuration, or hypdrophilicity/hydrophobicity. For purposes of example only, such substitutions could include substituting one aliphatic residue (Ile, Val, Leu, or Ala) for another, or substitution of basic residues Lys and Arg, acidic residues Glu and Asp, amide residues Gln and Asn, hydroxyl residues Ser and Tyr, or aromatic residues Phe and Tyr. Conservative variants can generally be identified by modifying a polypeptide sequence of the invention and evaluating the antigenic activity of the modified polypeptide using, for example, an immunohistochemical assay, an enzyme-linked immunosorbent assay (ELISA), a radioimmunoassay (RIA), or a western blot assay. Further information regarding the making of phenotypically silent amino acid exchanges may be found in Bowie et al., Science 247:1306-1310 (1990).

Other variants are also contemplated within the scope of the invention, and such variants include amino and/or carboxyl terminal fusions, for example achieved by addition of amino acid sequences of any number of residues, as well as intrasequence insertion of one or more amino acids. For example, amino acid sequences added may be those derived from the whole or parts of other polypeptides or proteins, or may be those provided in the corresponding positions in the FIV envelope or viral protein. Longer peptides may comprise multiple copies of one or more of the polypeptide sequences. Moreover, multiple copies of the polypeptides may be coupled to a polyamino acid backbone, such as a polylysine backbone to form multiple antigen peptides (MAPs).

Deletional amino acid sequence variants are those in which one or more amino acid residues are removed from the sequence. Insertional variants exist when one or more amino acids are integrated into a predetermined site in the polypeptide, although random insertion is an option with suitable screening of the resulting product. In all cases, these and other FIV variants used retain substantially the same antigenicity of the FIV polypeptides. Other variants are also contemplated, including those where the amino acid substitutions are made in the area outside the antibody recognition regions of the protein. Fusion proteins comprising two or more polypeptide sequences of FIV are also within the scope of the invention provided the sequences provide the appropriate antigenicity. Such polypeptides will generally correspond to at least one epitope or mimitope that is characteristic of FIV. By characteristic, it is meant that the epitope or mimitope will allow immunologic detection of antibody directed to FIV in a physiological sample with reasonable assurance. Usually, it will be desirable that the epitope or mimitope, variant or fusion protein be immunologically distinct from (i.e., not cross-reactive with antibodies which recognize) viruses other than FIV.

An antigenically active variant differs by about, for example, 1, 2, 3, 5, 6, 10, 15 or 20 amino acid residues from SEQ ID NOS: 1-3, or a fragment thereof. Where this comparison requires alignment the sequences are aligned for maximum homology. Deletions, insertions, substitutions, repeats, inversions or mismatches are considered differences. The differences are, preferably, differences or changes at a non-essential residue or a conservative substitution. The site of variation can occur anywhere in the polypeptide, as long as the resulting variant polypeptide is antigenically substantially similar to SEQ ID NOS: 1-3. Exemplary functionally-equivalent variants include those displaying 50% or more amino acid homology. Preferably, such homology is 60%, 70%, or greater than 80%. However, such variants may display a smaller percentage of homology overall and still fall within the scope of the invention where they have conserved regions of homology.

In some cases, one or more cysteine residues may be added to the termini of the polypeptides in order to facilitate specific carrier linkage or to permit disulphide bonding to mimic antigenic loops and thus increase the antigenicity. Moreover, a fatty acid or hydrophobic tail may be added to the peptides to facilitate incorporation into delivery vehicles and to increase antigenicity immunogenicity.

Polypeptides of the invention can also comprise fragments of SEQ ID NOS: 1-3. For example, fragments of polypeptides can comprise at least about 5, 6, 8, 10, 12, 15, 18, 20, 22, 24, or 26 contiguous amino acids of the polypeptides shown in SEQ ID NOS: 1-3.

The FIV polypeptides used as detection reagents may be natural, i.e., including the entire FIV protein or fragments thereof isolated from a natural source, or may be synthetic. The natural proteins may be isolated from the whole FIV virus by conventional techniques, such as affinity chromatography. Polyclonal or monoclonal antibodies may be used to prepare a suitable affinity column by well-known techniques.

Polypeptides that are immunologically cross-reactive with a natural FIV protein can be chemically synthesized. For example, polypeptides having fewer than about 100 amino acids, more usually fewer than about 80 amino acids, and typically fewer than about 50 amino acids, may be synthesized by the well-known Merrifield solid-phase synthesis method where amino acids are sequentially added to a growing chain. (Merrifield, 1963, J. Am. Chem. Soc., 85:2149-2156). Recombinant proteins can also be used. These proteins may be produced by expression in cultured cells of recombinant DNA molecules encoding a desired portion of the FIV genome. The portion of the FIV genome may itself be natural or synthetic, with natural genes obtainable from the isolated virus by conventional techniques. Of course, the genome of FIV is RNA, and it will be necessary to transcribe the natural RNA into DNA by conventional techniques employing reverse transcriptase. Polynucleotides may also be synthesized by well-known techniques. For example, short single-stranded DNA fragments may be prepared by the phosphoramidite method described by Beaucage and Carruthers, 1981, Tett. Letters 22:1859-1862. Double-stranded fragments may then be obtained either by synthesizing the complementary strand and then annealing the strands together under appropriate conditions, or by adding the complementary strand using DNA polymerase with an appropriate primer sequence.

The natural or synthetic DNA fragments coding for the desired FIV protein or fragment may be incorporated in a DNA construct capable of introduction to and expression in in vitro cell culture. Usually, the DNA constructs will be suitable for replication in a unicellular host, such as yeast or bacteria. They may also be intended for introduction and integration within the genome of cultured mammalian or other eukaryotic cells. DNA constructs prepared for introduction into bacteria or yeast will include a replication system recognized by the host, the FIV DNA fragment encoding the desired polypeptide product, transcriptional and translational initiation regulatory sequences joined to the 5′-end of the FIV DNA termination regulatory sequences joined to the 3′-end of the fragment. The transcriptional regulatory sequences will include a heterologous promoter that is recognized by the host. Conveniently, a variety of suitable expression vectors are commercially available for a number of hosts.

To be useful in the detection methods of the present invention, the polypeptides are obtained in a substantially pure form, that is, typically from about 50% w/w or more purity, substantially free of interfering proteins and contaminants. Preferably, the FIV polypeptides are isolated or synthesized in a purity of at least 80% w/w, and more preferably, in at least about 95% w/w purity. Using conventional protein purification techniques, homogeneous polypeptide compositions of at least about 99% w/w purity can be obtained. For example, the polypeptides may be purified by use of the antibodies described hereinafter using the immunoabsorbant affinity columns described hereinabove.

The method of the invention may be accomplished using immunoassay techniques well known to those of skill in the art, including, but not limited to, using microplates and lateral flow devices. In one embodiment, an FIV polypeptide is immobilized on a solid support at a distinct location. Detection of polypeptide-antibody complexes on the solid support can be by any means known in the art. For example, U.S. Pat. No. 5,726,010, which is incorporated herein by reference in its entirety, describes an example of a lateral flow device, the SNAP® immunoassay device (IDEXX Laboratories), useful in the present invention. Colloidal particle based tests can also be used, such as the commercially available WITNESS® FIV diagnostic test (Synbiotics Corporation, Lyon, France).

Immobilization of one or more analyte capture reagents, e.g., FIV polypeptides, onto a device or solid support is performed so that an analyte capture reagent will not be washed away by the sample, diluent and/or wash procedures. One or more analyte capture reagents can be attached to a surface by physical adsorption (i.e., without the use of chemical linkers) or by chemical binding (i.e., with the use of chemical linkers). Chemical binding can generate stronger attachment of specific binding substances on a surface and provide defined orientation and conformation of the surface-bound molecules.

Another embodiment of the invention provides a device that is suitable for a lateral flow assay. For example, a test sample is added to a flow matrix at a first region (a sample application zone). The test sample is carried in a fluid flow path by capillary action to a second region of the flow matrix where a label capable of binding and forming a first complex with an analyte in the test sample. The first complex is carried to a third region of the flow matrix where an FIV polypeptide is immobilized at a distinct location. A second complex is formed between an immobilized polypeptide and the first complex including the antibody from the sample. For example, a first complex comprising a gold sol particle and an FIV polypeptide bound to an FIV antibody will specifically bind and form a second complex with a second immobilized FIV protein or with a second antibody directed to feline antibodies. The label that is part of the second complex can be directly visualized.

In another aspect, the invention includes one or more labeled specific binding reagents that can be mixed with a test sample prior to application to a device for of the invention. In this case it is not necessary to have labeled specific binding reagents deposited and dried on a specific binding reagent pad in the device. A labeled specific binding reagent, whether added to a test sample or pre-deposited on the device, can be for example, a labeled FIV polypeptide that specifically binds an antibody for FIV.

Any or all of the above embodiments can be provided as a kit. In one particular example, such a kit would include a device complete with specific binding reagents (e.g., a non-immobilized labeled specific binding reagent and an immobilized analyte capture reagent) and wash reagent, as well as detector reagent and positive and negative control reagents, if desired or appropriate. In addition, other additives can be included, such as stabilizers, buffers, and the like. The relative amounts of the various reagents can be varied, to provide for concentrations in solution of the reagents that substantially optimize the sensitivity of the assay. Particularly, the reagents can be provided as dry powders, usually lyophilized, which on dissolution will provide for a reagent solution having the appropriate concentrations for combining with a sample.

An FIV polypeptide can be an immobilized analyte capture reagent in a reaction zone (solid phase). A second analyte capture reagent, i.e. a second FIV polypeptide, that has been conjugated to a label, can either be added to the sample before the sample is added to the device, or the second analyte capture reagent can be incorporated into the device. For example the labeled specific binding reagent can be deposited and dried on a fluid flow path that provides fluid communication between the sample application zone and the solid phase. Contact of the labeled specific binding reagent with the fluid sample results in dissolution of the labeled specific binging reagent.

The device may also include a liquid reagent that transports unbound material (e.g., unreacted fluid sample and unbound specific binding reagents) away from the reaction zone (solid phase). A liquid reagent can be a wash reagent and serve only to remove unbound material from the reaction zone, or it can include a detector reagent and serve to both remove unbound material and facilitate analyte detection. For example, in the case of a specific binding reagent conjugated to an enzyme, the detector reagent includes a substrate that produces a detectable signal upon reaction with the enzyme-antibody conjugate at the reactive zone. In the case of a labeled specific binding reagent conjugated to a radioactive, fluorescent, or light-absorbing molecule, the detector reagent acts merely as a wash solution facilitating detection of complex formation at the reactive zone by washing away unbound labeled reagent.

Two or more liquid reagents can be present in a device, for example, a device can comprise a liquid reagent that acts as a wash reagent and a liquid reagent that acts as a detector reagent and facilitates analyte detection.

A liquid reagent can further include a limited quantity of an “inhibitor”, i.e., a substance that blocks the development of the detectable end product. A limited quantity is an amount of inhibitor sufficient to block end product development until most or all excess, unbound material is transported away from the second region, at which time detectable end product is produced.

The following are provided for exemplification purposes only and are not intended to limit the scope of the invention described in broad terms above. All references cited in this disclosure are incorporated herein by reference.

### EXAMPLES

### Example 1

Microplate ELISA analysis was performed on serum collected from confirmed FIV negative and infected cats, and cats vaccinated with the FEL-O-VAX® FIV vaccine (Fort Dodge Animal Health, Fort Dodge Iowa). This vaccine is produced from multiple strains of the whole killed FIV virus. Vaccinated cats were sampled at 84 days post-vaccination.

Antibodies to the following env FIV polypeptides were detected. The polypeptide (protein) includes a N-terminal cysteine (C) for use in conjugation chemistry.

[SEQ ID NO:1]

CELGCNQNQFFCK

[SEQ ID NO:2]

CELGSNQNQFFSK

[SEQ ID NO:3]

ELGSNQNQFFSKVPPELWKRYNKSKSKSKSKNRWEWRPDFESEKC

The free peptides, or the polypeptides conjugated to Bovine Serum Albumin (BSA), were coated on microplate wells at 5 to 10 ug/ml in a buffered solution at pH 8. Protein binding sites on the microplate wells were then blocked with, for example, BSA and wells were overcoated with a buffered sucrose solution.

Serum samples were initially diluted 10-fold and from this initial dilution, a series of dilutions was prepared so that the concentration was FIV antibody was diluted by 2, 4, 8, 16, 36, 64, 128, 256, 512, 1024, 2048, and 4096 fold. The dilution buffer was PBS containing 50% fetal bovine serum.

The diluted samples were added to the wells and the plates were incubated at room temperature for either 5, 10 or 60 minutes. Following incubation, the microplates were washed with PBS/Tween. Commercially available goat Anti-(cat IgG):peroxidase conjugate diluted in 50% fetal bovine serum was added to the wells. The plates were incubated for another fifteen minutes at room temperature and washed a second time with PBS/Tween. Peroxidase substrate was added and the plates were incubated a third time for 10 minutes at room temperature. Peroxidase product (activity) was measured with a spectrophotometer.

FIGS. 1A-C show the affect of sample antibody concentration (dilution) on the ELISA signals for infected and vaccinated cats. This experiment used polypeptide SEQ ID NO. 3 coated to the microplate wells and the diluted samples were initially incubated in the wells for 5, 10 and 60 minutes. A₆₅₀ was measured. S-N is the sample signal minus a negative control signal. At high sample dilutions (low sample concentrations), an infected cat is detected as positive by the method whereas a vaccinated cat is not detected as positive. The difference in assay signal between an infected cat and a vaccinated cat is increased at any given sample dilution as sample incubation time is decreased from 60 minutes to 5 minutes, so shorter sample incubation times also favor the differential detection of an infected cat using this method.

FIGS. 2A-C show the ratio of the signal from an infected cat to the signal from a vaccinated cat for the indirect format ELISA method using 3 different polypeptides containing FIV env sequences (SEQ ID NOs: 1, 2 and 3) coated on the microtiter plates with initial sample incubation times of either 5, 10 or 60 minutes. Sample concentration is relative to the initial serum sample dilution of 10-fold, and from this initial dilution a series of dilutions was prepared so that the concentration of sample was diluted by 20-, 40-, 80-, 160-, and 320-fold. The lowest sample concentration (highest dilution) used was assigned an arbitrary value of 10, and increasing sample concentrations used were 20, 40, 80, 160, and 320 relative to this. As shown in FIGS. 1A-C, lower sample concentrations and shorter sample incubation times increased the difference (ratio) of assay signals for an infected cat compared to a vaccinated cat for all three of the polypeptides in FIG. 2.

These results demonstrate a difference in kinetic parameters for the antibody/antigen binding reaction for antibodies in serum from FIV-infected, not vaccinated cats compared to antibodies in serum from vaccinated, uninfected cats. Furthermore, by systemic manipulation of these kinetic parameters, an immunoassay for FIV Antibody can be optimized for differential detection of antibodies in infected cats and lack of detection of antibodies in vaccinated cats.

Although various specific embodiments of the present invention have been described herein, it is to be understood that the invention is not limited to those precise embodiments and that various changes or modifications can be affected therein by one skilled in the art without departing from the scope and spirit of the invention.

What is claimed is:

1. A method for detecting antibodies to Feline Immunodeficiency Virus (FIV) in a biological sample, the method comprising contacting the biological sample with an FIV env polypeptide and detecting whether the polypeptide substantially binds to an antibody in the sample, wherein the reaction conditions are optimized so that the method will detect FIV antibodies in a sample from animals that have been naturally infected but the method will not detect antibodies in a sample from animals that have been vaccinated.

2. The method of claim 1 wherein the FIV env polypeptide is bound to a solid phase.

3. The method of claim 1 wherein the method is optimized by diluting the sample.

4. The method of claim 1 wherein the method is optimized by adjusting the concentration of the polypeptide.

5. The method of claim 1 wherein the method is optimized by adjusting the temperature of the reaction.

6. The method of claim 1 wherein the method is optimized by adjusting the time of the reaction.

7. A method for detecting a FIV infection in an animal comprising:
(a) contacting the sample with a solid phase having an immobilized FIV env polypeptide;
(b) contacting the sample and the solid phase with a species specific IgG antibody conjugated to a label;
(c) detecting the label, thereby detecting a FIV infection in the animal;
wherein the method is optimized so that antibodies to FIV that are the animal's immune response to a vaccination cannot be detected.

8. The method of claim 7 wherein the method is optimized by diluting the sample.

9. The method of claim 7 wherein the method is optimized by adjusting the concentration of the labeled antibody.

10. The method of claim 7 wherein the method is optimized by adjusting the temperature of the incubation.
